(19) United States
(12) Patent Application Publication
(10) Pub. No.: US 2026/0078078 A1
(43) Pub. Date: Mar. 19, 2026

(54) CONVERSION OF MUCONOLACTONE TO BETA-KETOADIPATES AND METHODS THEREFOR

(71) Applicant: Alliance for Sustainable Energy, LLC, Golden, CO (US)

(72) Inventors: Gregg Tyler BECKHAM, Golden, CO (US); Nicholas A. RORRER, Golden, CO (US); Gloria ROSETTO, Golden, CO (US); Daniyal Ali KIANI, Denver, CO (US)

(21) Appl. No.: 19/328,491

(22) Filed: Sep. 15, 2025

Related U.S. Application Data
(60) Provisional application No. 63/694,336, filed on Sep. 13, 2024.

(51) Int. Cl. C07C 67/333

(52) U.S. Cl. CPC C07C 67/333

(57) ABSTRACT

Described herein are materials and methods to generate beta-ketoadipates cost-efficiently and with reduced environmental impact from muconolactone. Advantageously, the described beta-ketoadipates, including dimethyl beta-ketoadipates, are more stable than traditional βKA and can be produced with a reduction in the amount of salt byproducts, by as much as half.

### CROSS-REFERENCE TO RELATED APPLICATIONS

This application claims priority from U.S. Provisional Patent Application No. 63/694,336, filed on Sep. 13, 2024, the contents of which are incorporated herein by reference in their entirety.

### CONTRACTUAL ORIGIN

This invention was made with government support under Contract No. DE-AC36-08GO28308 awarded by the Department of Energy. The government has certain rights in the invention.

### BACKGROUND

Beta-ketoadipic acid (βKA) is a useful reactant in the formation of plastic materials, specifically as replacement for nylon 6,6 production and as a copolymer in polyethylene terephthalate (PET) and provides performance advantages over other materials in both cases. However, βKA is relatively unstable in its acid form as it readily decarboxylates into levulinic acid. To compensate, βKA is generally chlorinated to increase its stability, which requires the use of toxic reagents and upon reacting liberates HCl. It can be seen from the foregoing that there remains a need in the art for beta-ketoadipates that can be easily and cost effectively generated with higher stability for use in the generation of polymers.

### SUMMARY

Described herein are materials and methods to generate beta-ketoadipates cost-efficiently and with reduced environmental impact from muconolactone. Advantageously, the described beta-ketoadipates, including dimethyl beta-ketoadipates, are more stable than traditional βKA and can be produced with a reduction in the amount of salt byproducts, by as much as half.

In an aspect, provided is a method comprising: a) providing methyl muconolactone; and b) reacting the methyl muconolactone in the presence of a base and an alcohol, thereby generating a β-ketoadipate.

In an aspect, provided is a material having the formula:

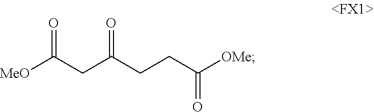 [SMILES: COC(=O)CCC(=O)CC(=O)OC]

wherein the material is generated from muconolactone.

In an aspect, provided is a catalyst comprising a copper oxide dispersed on a calcium oxide support.

In an aspect, provided is a method comprising: a) providing a calcium hydroxide support; b) dispersing copper nitrate trihydrate on the calcium hydroxide support via incipient wetness impregnation; and c) calcinating the copper nitrate trihydrate and calcium hydroxide support, thereby generating a copper oxide catalyst on a calcium oxide support.

For example, the β-ketoadipate may be dimethyl β-ketoadipate, as described in the formula <FX1>. The base may be, for example, NEt₃, pyridine, piperidine, N,N-dimethylamine, 1,5,7-triazabicyclo 4.4.0 dec-5-ene or a combination thereof. The alcohol may be methanol or one or more alcohol functional groups bonded to a C₂-C₁₂ straight chain, cyclic or aromatic group.

The described method may further comprise providing muconolactone; and reacting muconolactone in the presence of an acid and methanol, thereby generating the methyl muconolactone. The acid may be H₂SO₄.

The step of reacting may be performed in the presence of a solvent, including a polar solvent, for example, acetone, dichloromethane, tetrahydrofuran, or a combination thereof.

The catalyst may have a weight percentage of copper selected from the range of 1 wt % to 18 wt %, 2 wt % to 20 wt %, or optionally 5 wt % to 18 wt %. The catalyst may promote the conversion of methyl muconolactone to muconic acid at a conversion efficiency greater than or equal to 80%, 75%, 70%, 60% or optionally, 50%.

The step of calcinating is performed at a temperature of about 500° C. for about 4 hours, or at a temperature selected from the range of 400° C. to 600° C. or 450° C. to 550° C.

The described methods may also be performed using a K₂CO₃ or Na₂CO₃ catalyst.

### BRIEF DESCRIPTION OF DRAWINGS

Some embodiments are illustrated in referenced figures of the drawings. It is intended that the embodiments and figures disclosed herein are to be considered illustrative rather than limiting.

FIG. 1 provides an example schematic and reaction conditions for the generation of beta-ketoadipate including example components, as described herein.

FIG. 2 provides an example chemical mechanism using methanol as example for the generation of dimethyl beta-ketoadipate.

FIG. 3 provides representative NMR data of reaction products utilizing the described CuOₓ/CaO catalyst with reaction conditions of 40° C., 24 hr, 50 mg substrate, 50 mg catalyst, 2 ml H₂O.

FIG. 4 provides representative NMR data of reaction products utilizing the described CuOₓ/CaO catalyst with reaction conditions of 70° C., 24 hr, 50 mg substrate, 25 mg catalyst, 2 ml H₂O.

FIG. 5 provides representative NMR data of reaction products utilizing the described Na₂CO₃ catalyst with reaction conditions of 40° C., 24 hr, 50 mg substrate, 50 mg catalyst, 2 ml H₂O.

FIG. 6 provides representative NMR data of reaction products utilizing the described K₂CO₃ catalyst with reaction conditions of 40° C., 24 hr, 50 mg substrate, 50 mg catalyst, 2 ml H₂O.

### DETAILED DESCRIPTION

The embodiments described herein should not necessarily be construed as limited to addressing any of the particular problems or deficiencies discussed herein. References in the specification to “one embodiment”, “an embodiment”, “an example embodiment”, “some embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

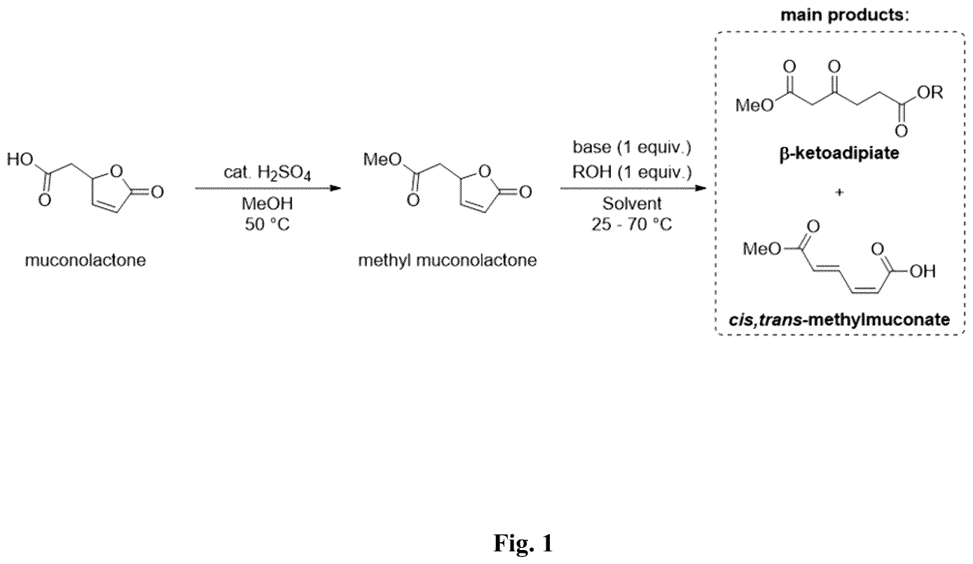

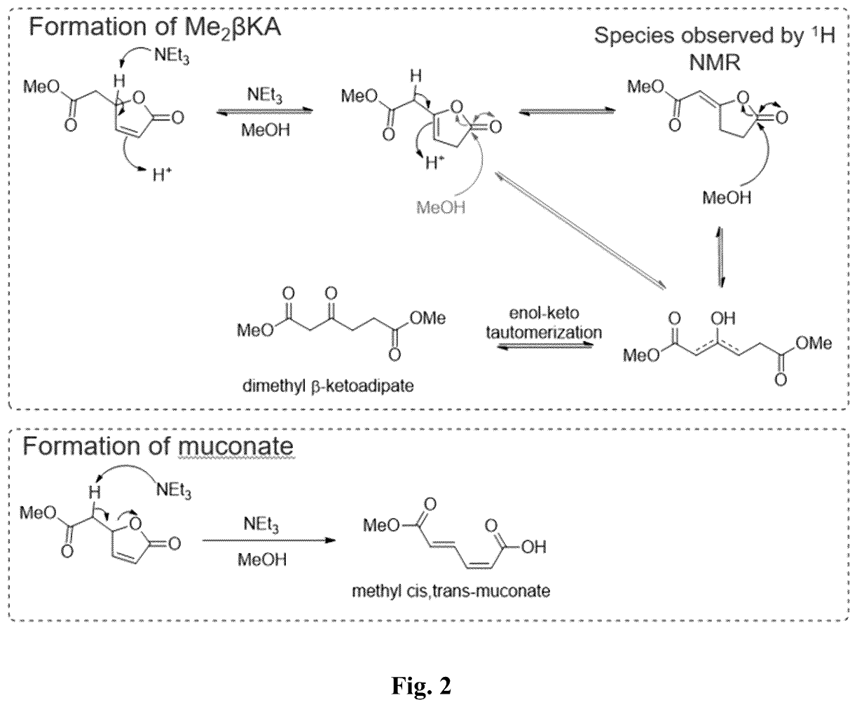

An example reaction with reactants, bases and solvents is provided in FIG. 1, with the proposed mechanism shown in FIG. 2.

### Example 1—CuOₓ/CaO Heterogeneous Catalyst

Described herein is a heterogeneous copper oxide supported on calcium oxide that is useful for the conversion of methyl muconolactone to muconic acid and methyl β-ketoadipate and can be precisely tuned based on reaction conditions. This catalyst may be useful with a wide range of reactants and products, including the conversion of any organic alcohol to branched products.

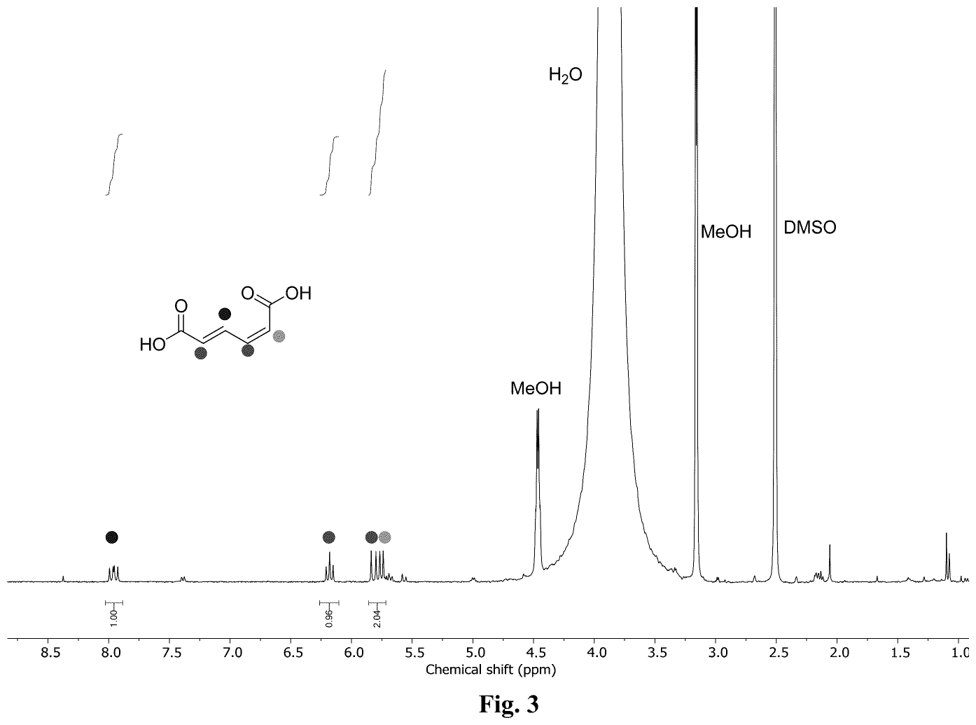

FIG. 3 shows NMR data for the conversion of methyl muconolactone to muconic acid. The reaction is performed at 40° C. for 24 hours with 50 mg CuOₓ and 50 mg CaO substrate in the presence of 2 ml of water. The use of CuOₓ/CaO relieves the burden of using homogeneous, stoichiometric base (e.g. K₂CO₃ or Na₂CO₃) and enables the same chemistry at comparable reaction rates using minuscule amounts of the solid catalyst that can be easily separated post reaction. The NMR data in FIG. 3 illustrates a 100% conversion of methyl muconolactone with 80% of product being cis/trans muconic acid and 20% isomer at 7.4 ppm. There is no apparent formation of methyl β-ketoadipate, illustrating 100% selectivity to muconic acid. The presence of methyl hydroxide suggests that the catalyst hydrolyzed the methyl ester to its acid form.

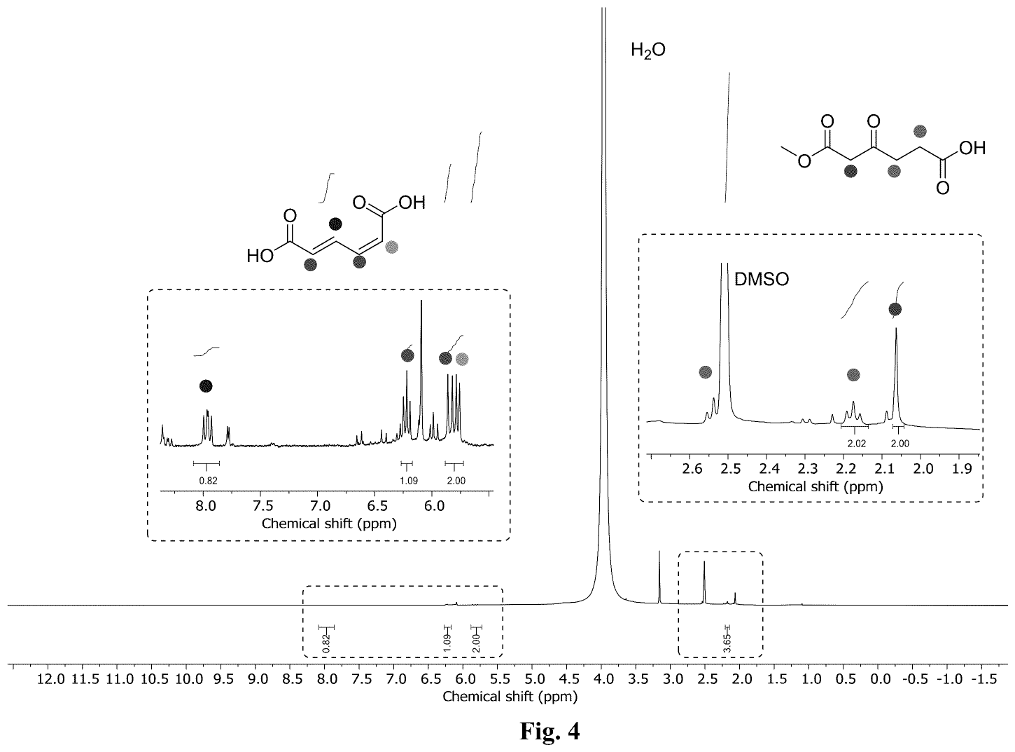

By increasing the reaction temperature, the described CuOₓ/CaO catalyst has increased selectivity towards methyl β-ketoadipate instead of muconic acid, as illustrated by FIG. 4. In this reaction, the temperature is increased to 70° C. from 40° C. and the catalyst loading is changed to 25 mg CaOₓ on a 50 mg substrate. The reaction time of 24 hours and 2 ml of water remain the same. FIG. 4 analysis shows that the 100% conversion of methyl muconolactone is preserved, resulting in 65% methyl β-ketoadipate with the remaining 35% product being cis/trans muconic acid/ester.

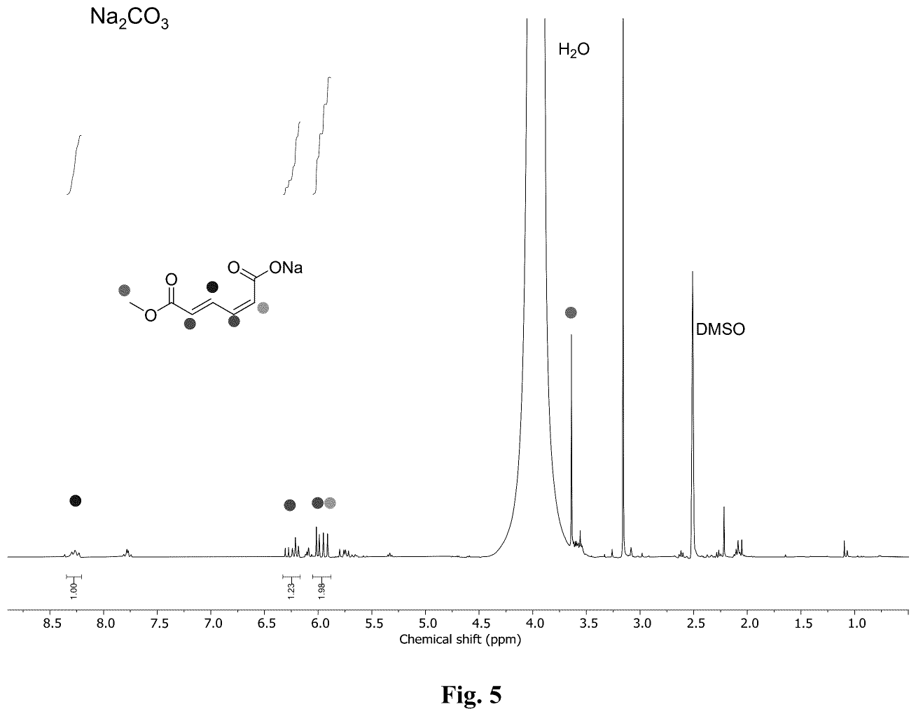

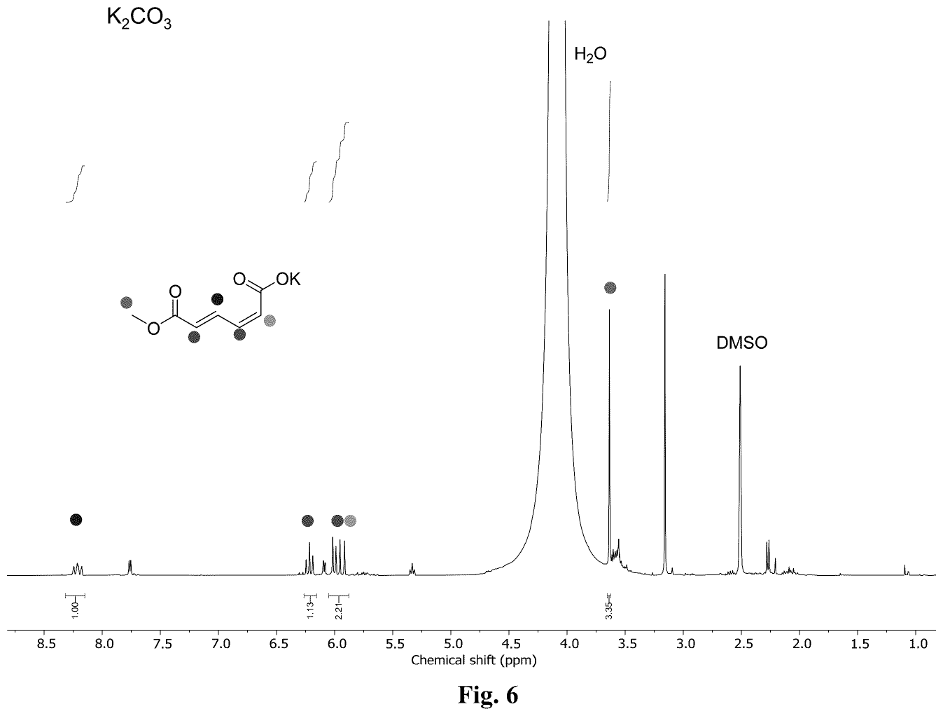

FIGS. 5 and 6 illustrate that the use of Na₂CO₃ and K₂CO₃, respectively, are also useful for the conversion of muconolactone at 40° C. for 24 hours. For Na₂CO₃ (FIG. 5), NMR analysis shows 70% conversion of muconolactone to muconic ester salt (based on their relative integrals). Other products present between 5.5 and 6.5 ppm. Beta ketoadipate triplet at 2.6 ppm, greater than 10% of muconic ester salt. For K₂CO₃ (FIG. 6), NMR shows 63% conversion of muconolactone to muconic ester salt with high selectivity of methyl muconate. However, no methyl β-ketoadipate is present.

### Catalyst Synthesis

An example catalyst composition constitutes 1-18% Cu by mass in the form of copper oxide dispersed on calcium hydroxide support. Copper nitrate trihydrate is used as the copper precursor and is introduced into the support via incipient-wetness-impregnation method. After drying at 120° C. the catalyst powder is then calcined at 500° C. for 4 hours, yielding an active, selective, and stable catalyst. At this calcination temperature, copper nitrate decomposes to copper oxide and calcium hydroxide transforms to calcium oxide, yielding a catalyst that we denote as W % CuOₓ/CaO where W % represents the weight percent loading of copper.

The present invention may be further understood from the following non-limiting examples:

Example 1. A method comprising:
providing methyl muconolactone; and
reacting the methyl muconolactone in the presence of a base and an alcohol, thereby generating a β-ketoadipate.

Example 2. The method of example 1, wherein the β-ketoadipate is dimethyl β-ketoadipate.

Example 3. The method of example 1 or 2, wherein the step of reacting is performed in the presence of a catalyst.

Example 4. The method of example 3, wherein the catalyst is a heterogenous catalyst comprising copper oxide and calcium oxide.

Example 5. The method of example 3, wherein the catalyst comprises K₂CO₃, NaCO₃ or a combination thereof.

Example 6. The method of any of examples 1-5 further comprising:
providing muconolactone; and
reacting muconolactone in the presence of an acid and methanol, thereby generating the methyl muconolactone.

Example 7. The method of example 6, wherein the acid is H₂SO₄.

Example 8. The method of any of examples 1-7, wherein the base is NEt₃, pyridine, piperidine, N,N-dimethylamine, 1,5,7-triazabicyclo 4.4.0 dec-5-ene or a combination thereof.

Example 9. The method of any of examples 1-8, wherein the alcohol is methanol.

Example 10. The method of any of examples 1-9, wherein the step of reacting methyl muconolactone further comprises reacting in the presence of a solvent.

Example 11. The method of example 10, wherein the solvent is a polar solvent.

Example 12. The method of example 10, wherein the solvent is acetone, dichloromethane, tetrahydrofuran, or a combination thereof.

Example 13. A material having the formula:

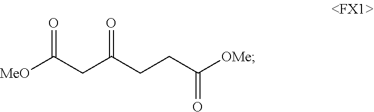 [SMILES: COC(=O)CCC(=O)CC(=O)OC]

wherein the material is generated from muconolactone.

Example 14. A catalyst comprising:
a copper oxide dispersed on a calcium oxide support.

Example 15. The catalyst of example 14, wherein the catalyst has a weight percentage of copper selected from the range of 1 wt % to 18 wt %.

Example 16. The catalyst of example 14 or 15, wherein the catalyst promotes the conversion of methyl muconolactone to muconic acid at a conversion efficiency greater than or equal to 75%.

Example 17. The catalyst of example 14 or 15, wherein the catalyst promotes the conversion of methyl muconolactone to β-ketoadipate at a conversion efficiency greater than or equal to 50%.

Example 18. A method comprising:
providing a calcium hydroxide support;
dispersing copper nitrate trihydrate on the calcium hydroxide support via incipient wetness impregnation;
calcinating the copper nitrate trihydrate and calcium hydroxide support, thereby generating a copper oxide catalyst on a calcium oxide support.

Example 19. The method of example 18, wherein the step of calcinating is performed at a temperature of about 500° C. for about 4 hours.

Example 20. The method of example 18 or 19, wherein the copper oxide catalyst has a weight percentage of copper selected from the range of 1 wt % to 18 wt %.

The provided discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure. For example, when a device is set forth disclosing a range of materials, device components, and/or device configurations, the description is intended to include specific reference of each combination and/or variation corresponding to the disclosed range.

Every formulation or combination of components described or exemplified herein can be used to practice the invention, unless otherwise stated.

Whenever a range is given in the specification, for example, a density range, a number range, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method comprising:
providing methyl muconolactone; and
reacting the methyl muconolactone in the presence of a base and an alcohol, thereby generating a β-ketoadipate.

2. The method of claim 1, wherein the β-ketoadipate is dimethyl β-ketoadipate.

3. The method of claim 1, wherein the step of reacting is performed in the presence of a catalyst.

4. The method of claim 3, wherein the catalyst is a heterogenous catalyst comprising copper oxide and calcium oxide.

5. The method of claim 3, wherein the catalyst comprises K₂CO₃, NaCO₃ or a combination thereof.

6. The method of claim 1 further comprising:
providing muconolactone; and
reacting muconolactone in the presence of an acid and methanol, thereby generating the methyl muconolactone.

7. The method of claim 6, wherein the acid is H₂SO₄.

8. The method of claim 1, wherein the base is NEt₃, pyridine, piperidine, N,N-dimethylamine, 1,5,7-triazabicyclo 4.4.0 dec-5-ene or a combination thereof.

9. The method of claim 1, wherein the alcohol is methanol.

10. The method of claim 1, wherein the step of reacting methyl muconolactone further comprises reacting in the presence of a solvent.

11. The method of claim 10, wherein the solvent is a polar solvent.

12. The method of claim 10, wherein the solvent is acetone, dichloromethane, tetrahydrofuran, or a combination thereof.

13. A catalyst comprising:
a copper oxide dispersed on a calcium oxide support.

14. The catalyst of claim 13, wherein the catalyst has a weight percentage of copper selected from the range of 1 wt % to 18 wt %.

15. The catalyst of claim 13, wherein the catalyst promotes the conversion of methyl muconolactone to muconic acid at a conversion efficiency greater than or equal to 75%.

16. The catalyst of claim 13, wherein the catalyst promotes the conversion of methyl muconolactone to β-ketoadipate at a conversion efficiency greater than or equal to 50%.

17. A method comprising:
providing a calcium hydroxide support;
dispersing copper nitrate trihydrate on the calcium hydroxide support via incipient wetness impregnation;
calcinating the copper nitrate trihydrate and calcium hydroxide support, thereby generating a copper oxide catalyst on a calcium oxide support.

18. The method of claim 17, wherein the step of calcinating is performed at a temperature of about 500° C. for about 4 hours.

19. The method of claim 17, wherein the copper oxide catalyst has a weight percentage of copper selected from the range of 1 wt % to 18 wt %.
